(19) United States
(12) Patent Application Publication
(10) Pub. No.: US 2010/0196289 A1
(43) Pub. Date: Aug. 5, 2010

(54) DOUBLE LAYERED OXIDE AND HYDROXIDE PIGMENTS AND METHOD OF THEIR PREPARATION

(76) Inventor: Carlo Ghisalberti, Sao Paulo (BR)

Correspondence Address: HESS PATENT LAW FIRM, P.C., 9 MIRAMAR LANE, STAMFORD, CT, 06902, US

(21) Appl. No.: 11/993,221

(22) PCT Filed: Dec. 13, 2005
(86) PCT No.: PCT/IB05/03744

§ 371 Date: Feb. 20, 2008

(30) Foreign Application Priority Data
Jun. 23, 2005 (IT) MI2005A001186

(51) Int. Cl. A61K 8/58; A61Q 1/02; A61Q 1/06; A61Q 1/10; C08G 77/398; C07F 15/02; C07F 7/28; A61Q 1/12; A61Q 17/04

(52) U.S. Cl. 424/59; 424/63; 424/64; 424/70.7; 514/772.1; 514/784; 525/475; 549/212; 556/9; 556/14; 556/27; 556/31

(57) ABSTRACT

A pigment having at least two layers according to the following structure (I), where C is an internal pigment core of formula [M^{x}(O)_{y}(OH)_{z}]; where M^{X}=Ti^{IV}, Zn^{II}, Zr^{IV}, Sn^{IV}, Fe^{II}, Fe^{III}, Sn^{II}, Cr^{VI}, or Mn^{II}; x=2, 3 or 4; and (y/2+z)=x; A is a plant polyphenol having an active-redox, radical scavanger behaviour and at least two chelating moieties and B is an outer hydrophobic layer consisting in an anionic surfactant, and where Mₐ and M_{b} denotes, each independently, M^{q+}=Ca²⁺, Zn²⁺, Ti⁴⁺, Si⁴+, Al³⁺, Sn⁴⁺, Mg²⁺, or Sr²⁺; and a=0 or 1; b=1; q=2, 3 or 4.

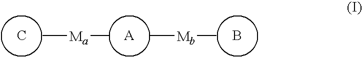

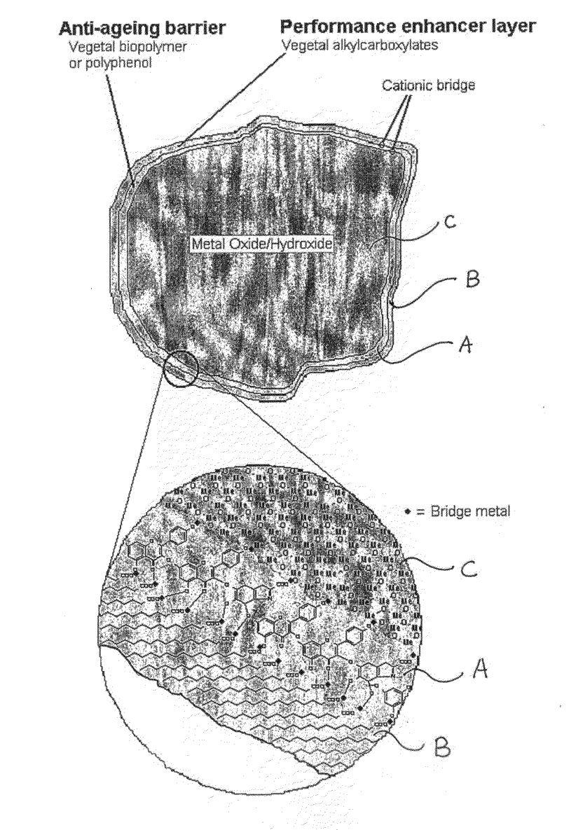

### FIELD OF THE INVENTION

The present invention refers to double-layered metal oxide and hydroxide pigments and to a method of their preparation. More particularly, the invention relates to metal oxide and hydroxyde pigments comprising a core of inorganic metal oxides and hydrated oxides, an inner layer of redox-active plant polyphenols and an outer layer of hydrophobic substances.

The present invention also refers to a “one-pot” method of producing inorganic double-layered pigments carried out in aqueous media by a sequential treatment which afford the above mentioned inner layer with anti-radical behaviour and the outer layer with hydrophobic properties.

The invention further relates to cosmetic formulations such as decorative (“make-up”) and photo-protective (“sun products”) compositions, as well as to pharmaceutical and food products comprising said pigments.

### BACKGROUND OF THE INVENTION

The oxides and hydrated oxides of transition metals as micron or sub-micron powders are widely applied for their pigmenting properties in a variety of technical applications.

However, these metal compounds display catalytic redox properties: they can promote oxidative stress on skin by activating Fenton-like reactions to generate harmful free oxygenated radicals. The production of these Reactive Oxygenated Species (ROS) is of particular relevance in the direct application on skin such as in the cosmetic art.

In fact, the ultrafine powders made with transition metal used in make-up applications and inorganic sunscreen display photodynamic properties which facilitate the electronic transfer to form ROS, thereby leading to the photo-oxidative damages.

The contact of transition metal oxides promoting oxidative burdens and skin photo-aging was early described by Jurkiewicz B. A. and Buettner G. R. in Photochem Photobiol, 64(6):918-22 (1996). A recent work by Brezova V. et all. in J. Photochem. Photobiol. E: Biology; (79) 121-134 (2005) also pointed out that the photo-excitation of commercial sun cream containing titanium dioxide promotes the formation of ROS.

Lipophilic coating of pigments, i.e. surface modification of pigment oxide and hydrated oxides with organic compounds, is known and has been found effective in improving water repellency, adhesion to the skin, and dispersion properties in various color cosmetic products. Lipophilic coating was disclosed in several patents, with silicones in U.S. Pat. No. 5,458,681, hydrogenated lecithin in U.S. Pat. No. 4,622,074, with fatty soaps in U.S. Pat. No. 4,648,908, silicone-containing compound and fatty acid ester in U.S. Pat. No. 5,738,841, hydrogenated lecithin in U.S. Pat. No. 4,622,074, in U.S. Pat. No. 6,080,424 by natural phospholipid and proteins. Noteworthy, such lipidic surface treatments are mainly focused on the improvement of the workability in the manufacturing of the cosmetic formulations and the performances of the finished products at end-user level. as a matter of fact, they have no appreciable effect on the ROS production problems.

In our previous application (WO01/55263) we have tested the possibility to improve the photodynamic behaviours of the micronized inorganic oxides and hydroxides by a surface treatment with polyphenolic substances. However the resulting pigments have poor performance with regard to workability in the manufacturing and to end-users performances; these problems could not be overcome by a post-coating lipophilic treatment which resulted in a mixed layer with intermediate, insufficient properties in both antioxidant passivation and hydrophobic behaviour.

Therefore, there is the need of a novel pigment system which is passivated with respect to the photo-oxidative catalysis, otherwise promoted by transition metal compounds, particularly in composition intended for the direct contact with skin such as cosmetic and anti-UV compositions, whose photo-ageing action must be minimized.

It is an aim of the present invention to provide a pigment that solves the above mentioned problems and that can be manufactured in a cost-effective way.

### SUMMARY OF THE INVENTION

The above aim is reached by means of the present invention, that relates to an inorganic pigment according to claim 1. the invention also relates a method of manufacturing such pigment according to claim 18 and to a cosmetic product incorporating said pigment.

The present invention refers to pigments comprising a pigment core of inorganic oxides and hydroxides (known per se) that is linked, directly or through a polyvalent cation to one or more plant polyphenols having at least two chelating functions, said plant polyphenols being linked through a further polyvalent cation to an anionic surfactant. In other words, the plant polyphenol is forming a first inner layer around the core and the surfactant is forming a second outer layer, thus providing a double layer pigment. It is within the scope of the invention also a pigment comprising further layers connected to the outer layer.

The inner molecular layer prevents the photo-oxidative damage of transition metal oxides, so that the skin damage resulting from the combination of UV-radiation, atmospheric oxygen and metal catalysis (Fenton-like reactions) is actually inhibited.

The outer molecular layer provides pigments suitable for make-up formulations and high-SPF sun products, i.e. containing inorganic sunblockers, thereby characterized by excellent performances in industrial manufacturing along with the prevention of the photo-oxidative burden on skin.

These pigments are finished with an outer hydrophobic layer, chemically bonded to the first inner layer, which is dispersible in the lipid carriers employed in the manufacturing of decorative and sun blockers formulations.

Moreover, the use of the pigments of invention as sunblockers avoid the whitish effect of the formulations comprising the micronized ZnO or TiO₂, i.e. with similar performances of the corresponding nanopigments, yet without the risk of percutaneous absorption.

The present pigment and their manufacturing method provide a novel, highly effective surface inactivation inorganic oxides and hydroxides with specific anti-photoaging barrier.

### DETAILED DESCRIPTION OF THE INVENTION

As shown in following general formulas and in the enclosed FIGURE, the pigments of invention comprise a core C of inorganic metal oxides and hydroxides, a first inner layer A of polyphenolic, redox-active substance that is bonded to the core and a second outer layer B of hydrophobic substances that are bonded to the first inner layer. Layers A and B are bonded through a metal cation acting as a bridge; core C and layer A can be bonded through said cationic bridge or directly (a=0).

In detail, the pigments of the present invention have the following structure (I):

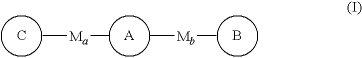

wherein:
C denotes the internal core of formula [M^{x}(O)_{y}(OH)_{z}]; where M^{x} is one or more of Ti^{IV}, Zn^{II}, Zr^{IV}, Sn^{IV}, Ce^{IV}, Fe^{II}; Fe^{III}; Sn^{II}; Cr^{VI}, Mn^{II} x=2, 3 or 4; and (y/2+z)=x;
said C having a diameter comprised between 20 and 0.02 μm;
A denotes the first, inner, layer constituted of a plant polyphenolic substance having an active-redox, radical scavanger behaviour and at least two chelating moieties;
B denotes the outer hydrophobic layer constituted of one or more anionic surfactant;
Mₐ and M_{b} denotes, each independently, M^{q+};
M^{q+} is selected from Ca²⁺, Zn²⁺, Ti⁴⁺, Si⁴⁺, Al³⁺, Sn⁴⁺, Mg²⁺, or Sr²⁺;
   a ═0 or 1; b=1; q=2, 3 or 4;
C is selected from:
   Cᵢ: white oxides and hydrated oxides of Ti^{IV}, Zn^{II}, Zr^{IV, Sn}^{IV}, Ce^{IV, Zr}^{IV};
   Cᵢᵢ: coloured oxides and hydrated oxide of Fe^{II}, Fe^{III}; Sn^{II}; Cr^{IV}, Mn^{II};
A is selected from:
   Aᵢ: polymeric polyphenols;
   Aᵢᵢ: anthocyanins or flavonoids;
   Aᵢᵢᵢ: non-flavonoid plant polyphenols;
B is selected from:
   Bᵢ: carboxylic-ended surfactants;
   Bᵢᵢ: phosphoric-ended surfactants;
   Bᵢᵢᵢ: sulfonic-ended surfactants;
   Bᵢᵥ: perfluorurated surfactants;
   Bᵥ: siloxane chelating surfactants.

According to a preferred embodiment the amount of A is 1-4% (w/w) of C, preferably 1-2% (w/w); the amount of B is 3-8% (w/w) of C, preferably 3-5% (w/w). AB is within the range of 0.05 to 2 and preferably is within the range of 0.2 to 0.4. The above ratios are by weight.

The pigments according the invention have a core C (i.e. the pigment before being treated according to the invention method) consisting of metal oxides and hydroxides, with size from 2 to 0.02 μm, preferably from 0.5 to 0.05 μm. The pigments C are selected from white (Cᵢ) and colored (Cᵢᵢ) pigments and mixtures thereof; the core can also contain two or more metal oxides or hydroxides.

Suitable Cᵢ include: titanium dioxide and oxide hydrated (TiO₂, Ti(OH)₂, rutile and anatase, zinc oxide (ZnO), anhydrous and hydrated alumina (Al₂O₃, AlOOH), tin^{IV} oxide (SnO₂), cerium oxide (CeO₄), and zirconium dioxide (ZrO₂).

Suitable Cᵢᵢ include: iron oxides and hydroxides such as FeO (yellow), Fe₂O₃ and Fe₂O₃.H₂O (red), FeₓO_{y} (brown), Fe₃O₄ (black); chromium oxides and hydroxides (Cr₂O₃.nH₂O and Cr₂O₃); tin^{II} oxide (SnO), manganese oxide (MnO).

Also included are hybrid pigments formed by the combination of two or more metal cations from Cᵢ or from Cᵢᵢ, hybrid pigments formed by the combination of Cᵢ and Cᵢᵢ, e.g. TiO₂/Fe₂O₃, SiO₄/TiO₂, Sia₄/Fe₂O₃, Al₂O₃/TiO₂, as well as pigments and nacres formed by silicates, alumosilicates, borates, clay, talc, mica, sericite which are mixed or are surface-treated with the oxides and hydrated oxides previously mentioned.

Preferred Cᵢ are titanium dioxide (TiO₂, rutile and anatase) and zinc oxide (ZnO).

Preferred Cᵢᵢ are iron oxides and hydroxides such as FeO (yellow), Fe₂O₃ and Fe₂O₃.H₂O (red), FeₓO_{y} (brown) and Fe₃O₄ (black).

The pigments according to the invention possess an inner layer A formed with plant polyphenol having radical scavenging and redox-active properties, i.e. are able to quench or capture the ROS promoted by the photo-catalytic action of C in the presence of light and atmospheric oxygen (air). Said substances shall further have at least two chelating moieties in order to link both the core C and the outer hydrophobic layer B.

Suitable Aᵢ include: plant melanin and tannins, i.e. vegetal biopolymers of high molecular weight, i.e. comprised between 1000 and 6000 dalton and up to 30000 dalton comprising carbonyl and phenolic moieties (optionally oxidated to semiquinones and quinones) and a variety of chelating centers e.g. phenolic group having proximal or vicinal phenolic and/or carbonyl groups.

The tannin suitable for our purposes are both hydrolysable and non-hydrolysable tannins. Based on the classification of Khanbabaee e van Ree (Nat. Prod. Rep., 2001, 18:641-649) the former are gallo/ellagitannins or complex tannins, the latter are condensed tannins.

Hydrolysable tannins are preferably obtained from Rhus semialata e Quercus infectoria (chinese or turk galls), Caesalpinia spinosa (Tara); condensed tannins from Vitis vinifera (grape seed), Schinopsis spp. (quebracho), Acacia catechu (catechu) or Acacia mollisima (mimosa), Rhizophora (mangrovia), Eucalyptus or Uncaria gambler; whilst mixed tannins are obtained from Castanea sativa (chestnut) or Rhus semialata (sumac).

Suitable vegetal may be either extracted from plant sources, as disclosed in WO00/09616, or more conveniently by synthesis from vegetal monomers or in combination with eumelanin precursors, the phytomelanins disclosed in WO01/018125.

In a preferred embodiment of the invention A′ is a hybrid polymeric polyphenol, i.e. obtained by oxidative copolymerization of a alkaline solution of tannins and eumelanin precursors, typically L-dopa.

Suitable Aᵢᵢ are anthocyanins or flavonoids, either in the free form (“aglycones”) or glycosilated and/or partially O-acetylated or O-methylated.

The anthocyanins appear in the petals of flowers, in the leaves of many plants and in the fruit of colored fruits and vegetables, in skins (egg plant, apple), in the fruit body (cherry, blackberry), or in peel and pulp of grape. The chemistry of these materials is based on 2-phenylbenzopyrylium (flavylium) substituted with hydroxy or methoxy groups, namely pelargonidin, cyanidin, delphinidin, petunidin, peonidin, malvidin, which may also be mono, di and tri-saccharides, and position 3 may be acylated, eg with p-coumaric acid.

Flavonoides include a large structural variety of compounds such as: flavandiols (e.g. dihydroquercetin), flavanonols (e.g. miricetin), flavones (e.g. luteolin), flavonols (e.g. quempferol, morin and quercetin), flavanones (e.g. esperetin), flavones (e.g. naringenin), flavononols (fisetin) and flavanonols (fustine), including glycosides and esters thereof.

Illustrative examples include the generally referred as “citrus bioflavonoids” because of their high content in Citrus spp e.g. hesperetin, naringenin, eriodictyol, diosmin and glycosides thereof, typically found in citrus peels; examples of flavones include as luteolin, apigenin and crysin and glycosides thereof. Mixed flavonoids occurs in a wide range of natural materials, including fruit such as apple peel, pears, bell peppers, lemons, tomatoes, olives; and vegetables including radishes (Saxa treib), kohlrabi, horseradish. Other examples of mixed flavonoids are found in Chrysanthemum and goldenrod; gardenins from Gardenia l., etc.

Preferred Aᵢᵢ are grape anthocyanes, rutin and citrus bioflavonoids.

Aᵢᵢᵢ are non-flavonoid polyphenols with molecular weight <1000 dalton. The term “non-flavonoid polyphenols” as used herein include plant-derived aromatic substance with non flavonoid structure comprising at least a proximal and/or vicinal carbonyl and phenolic pair. Said groups represents both chelating centers to link C to B and the redox-active groups capable of scavenging the ROS generated by C in the photo-oxidative catalyzes.

Aᵢᵢᵢ are selected among plant substances with the following structures: phenolic acid and dimers (2×C₆₊₁), coumarinic and isocoumarinic (C₆₊₃), chromonic (C₆₊₃), naphthoquinonic (C₆₊₄), anthraquinonic (C₆₊₂₊₆), xhantonic (C₆₊₁₊₆), stylbenic (C₆₊₂₊₆), neolignanic (C₆₊₃)₂.

Exemplary, Aᵢᵢᵢ for our purposes may include hydroxylated and/or quinones substances obtainable from vegetals, such as gallic acid, ellagic acid, curcumin, plant anthraquinones (e.g. rhein, aloe-emodin, frangulin, crysofanol, cascarosides, morindon), plant naphtoquinones (e.g. alkannin, shikonin, lapachol), and the like.

The pigments according to the invention have an outer layer B wherein B is an anionic surfactant, which are capable of chelating the partially chelated, polyvalent cation M_{b} provided within the inner layer A.

Anionic surfactants suitable for the purpose of the invention are the commercially available anionic surfactants, sold either as salt or in non-ionized form.

Suitable Bᵢ include: carboxylate of formula ⁽⁻⁾OOC—R; dicarboxylate of formula ⁽⁻⁾OOC—R—OOC⁽⁻⁾; α-hydroxycarboxylate of formula ⁽⁻⁾OOC—CHR′—O—CO—R; alkylsuccinamate of formula ⁽⁻⁾OOC—CH₂CH₂—O—CO—R; N-alkanoylsarcosinate or glycinate of formula ⁽⁻⁾OOC—CH₂—NR′—CO—R; N-acylglutamate of formula ⁽⁻⁾OOC—CH₂CH₂CH(NH—CO—R)—COO⁽⁻⁾; N-acylaspartate of formula ⁽⁻⁾OOC—CH₂CH(NH—CO—R)—COO⁽⁻⁾; and Nε-acyl-lysinate of formula ⁽⁻⁾OOC—CH(NH₂)—(CH₂)₄—NH—CO—R.

Suitable Bᵢᵢ include: alkyl o dialkyl-phosphate of formula: ⁽⁻⁾OₐP(O)—(OR)_{b}; wherein a=−1 or 2; and b=3−a.

Suitable Bᵢᵢ include: alkyl sulfonate of formula ⁽²⁻⁾O₃S—R; alkylsulfate of formula ⁽²⁻⁾O₃S—O—R; alkyl ester sulfonate of formula: ⁽²⁻⁾O₃S—CHR—COOR′; alkyl amide sulfate of formula ⁽²⁻⁾O₃SO—NHR′—CO—R; alkyl-isethionate of formula: ⁽²⁻⁾O₃S—CH₂CH₂—CO—R; N-acyl-N-alkyltaurate of formula ⁽²⁻⁾O₃S—CH₂CH₂—NR′—CO—R; C₉-C₂₀ alkylbenzene-sulfonate; alkylsulfosuccinate of formula ⁽²⁻⁾O₃S—CH(COOR′)—CH₂—R sulfosuccinate monoesters or diesters; alkylglycosides sulphate, and the like.

In Bᵢ, Bᵢᵢ and Buᵢᵢᵢ, each —R or —C—OR radical is a saturated or unsaturated, linear or branched, alkyl or acyl C₂-C₂₂, preferably C₈-C₁₈; said R optionally comprising a polyalkoxy chain of formula —(OCH₂CHR′)_{z}—O— with z comprised between 1 and 100, preferably between 1 and 10; and each R′ is H or C₁-C₃ alkyl, preferably Cᵢ.

Preferred —R or —C—OR radicals include those derived from capric, caprylic, capric, lauric, myristic, palmitic, stearic, isostearic, sebacic, palmitoleic, linoleic acids and alcohols, alone or in combination, e.g. from hydrolyzed vegetal oils.

Suitable Bᵢᵥ include: perfluoroalkylcarboxylate of formula ⁽⁻⁾OOC—R″, perfluoro-alkyl or dialkyl-phosphate of formula: ⁽⁻⁾OₐP(O)—(OR″)_{b}; a=1 or 2; b=3; perfluoroalkylsulfonate of formula ⁽⁻⁾O₃S—R″; wherein R″ is a fluorocarburic C₄-C₂₀ chain, preferably C₈-C₁₈;

Suitable Bᵥ include: silicone polymers comprising a hydrophobic polysiloxane backbone and at least one metal binding site covalently bound to the hydrophobic polysiloxane backbone, e.g. those disclosed in U.S. Pat. No. 6,566,322 and in “Silicone Surfactants” edited by Randal M. Hill. in the 3^{rd} Chapter “Novel Siloxane Surfactant Structures”.

Each specific B offers distinctive performances, e.g. increases holding moisture capacity; reduce stickiness, increase spreading; enhances smooth feel and adhesion to skin; increases refreshment and/or smoothness to the cosmetic comprising thereof.

Preferably the surfactants are employed as alkaline salts (e.g. sodium and potassium salts) or ammonium salts (e.g. ammonium, MEA, and DEA salts) to facilitate the ionic exchange with the residual polyvalent cations M during the pigment manufacturing, as further detailed in the Examples section.

In the pigment of invention, the optional linker Mₐ and M_{b}, are polyvalent cation M^{q+}. Suitable polyvalent cations are selected among water soluble salts either in cationic or in amphoteric form; e.g. sulfates, nitrates, chlorides and acetates of Al³⁺, Zn²⁺, Mg²⁺, Ca²⁺, Sr²⁺ and Ba²⁺ salts; or alkaline soluble salts and amphoteric complexes of Zn²⁺, Ti⁴⁺, Al³⁺, Sn⁴⁺ such as Na or K aluminate, zincate and silicate.

Mₐ and M_{b} can be the same or different.

These polyvalent cations can be fully combined with the chelating moieties of A and B, or may contain a residual monovalent anion X⁽⁻⁾, such as OH⁽⁻⁾ or Cl⁽⁻⁾ when M^{q+}_{a,b} are a polyvalent cation with q higher than 3, such as Al³⁺, Ti⁴⁺, Si⁴⁺ and the like.

As for the method of manufacturing the pigments of invention, the general method is based on wet-chemical coating of the core pigments in a one-pot process, wherein the metal oxides and hydroxides are suspended in water, and their reaction with an alkaline solution of A and of B is carried out in one step.

Although many variations can be conceived, the application of the method of invention end up in two main versions, according to the structure of the inner layer compound.

The first is a “C-Mₐ-A-M_{b}-B” pigment, i.e. with Mₐ (a=1), meaning that the inner layer is linked to the core (C) via a cationic bridge as depicted in the schematic structure (I-a):

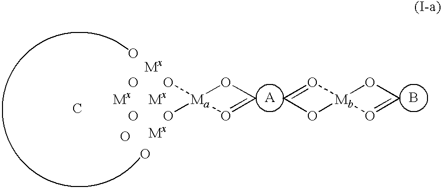

The method according to this embodiment comprises the steps of suspending core C in water at about 80-90° C., adding to the suspension Mₐ in the form of a salt solution (ampho salt or cationic salt), adding A⁽⁻⁾ salt solution adding M_{b} and eventually adding B⁽⁻⁾.

The second is a “C-A-M_{b}-B” pigment, i.e. without Mₐ (a=0), meaning that the inner layer is directly linked to the core (C) as depicted in the schematic structure (I-b):

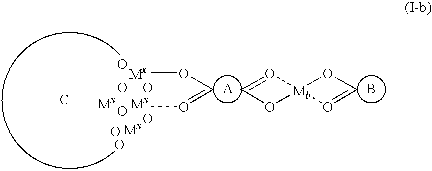

The method is similar to the above one, but there is no addition of Mₐ before reacting with A⁽⁻⁾ solution.

The “C-Mₐ-A-M_{b}-B” pigments are typically afforded by the Methods I and II, whilst the “C-A-M_{b}-B” pigments by the Method II, as detailed in the Examples section.

The pigments obtained as an aqueous slurry may be isolated in dry form by conventional drying methods, e.g. filtration, spray drying, jet-milling or fluidized bed drying

Alternatively, the aqueous slurry may washed up from the salts formed during the sequential reactions, and the pigments can be used as an aqueous or lipidic suspension, optionally with the addition of a 1 to 10% pigment dispersants.

The invention also relates to cosmetic or dermatological compositions comprising the pigments described above, which may also comprise any cosmetically acceptable ingredients. The expression “cosmetically acceptable ingredients” designate includes the material of INCI list drawn by the European Cosmetic Toiletry and Perfumery Association (COLIPA) and issued in 96/335/EC “Annex to Commission Decision of 8 May 1996”.

Cosmetically acceptable ingredients include oils, waxes, surfactants, silicones, perfluorides, synthetic organic UV-absorbers, fragrances or other materials listed in INCI.

The composition may also contain other non-coated or differently coated powder, as well as nacres and/or fillers, and also other pigments that are well known in the art.

The nacres may be present in the composition in a proportion of from 0 to 20% by weight and preferably from 8% to 15% by weight, and may be chosen from natural mother-of-pearl, mica coated with titanium oxide, with iron oxide, with natural pigment or with bismuth oxychloride, and also coloured titanium mica.

The fillers, which may be present in a proportion of from 0 to 30% by weight and preferably from 5% to 15%, in the composition, may be mineral or synthetic, and lamellar or non-lamellar. Mention may be made of talc, silica, kaolin, nylon powder, PE powder, Teflon, starch, boron nitride, polymer microspheres, silicone microbeads, muscovite, sericite, Mg and Ca carbonate, silicates, clay, BiOCl, or beads of a polymeric material and metal soaps, as well as other treated or untreated pigments.

The pigments of the invention can be employed alone or in combination with others pigments and fillers in cosmetic, such as make-up and sun protection, compositions in any amounts between 0.1 and 70% by weight. One or more kind of the pigments, according to the different possible solutions of the invention, may be present in said compositions.

Typical make-up compositions are eye shadow pencils (pigment content from about 5 to 15%), mascara (pigment content from about 10 to 70%), eye shadow powder compacts (pigment content from about 20 to 70%), liquid compositions for eye shadow and eye makeup (pigment content from about 7 to 15%), lipsticks and lip gloss (pigment content from about 10 to 20%), make-up in pencil form (pigment content from about 15 to 25%), make-up powder compacts (pigment content from about 10 to 50%), make-up emulsions (pigment content from about 5 to 10%), make-up gel (pigment content from about 1 to 5%), foam bath concentrates with color gloss (pigment content from about 0.1 to 2%), skin care lotions (pigment content from about 0.1 to 2%), sun-protecting emulsions and lotions (pigment content from about 1 to 20%).

The composition may also comprise a water-soluble or liposoluble colorant, especially a natural organic colorant such as cochineal carmine, and/or a synthetic colorant such as halo acid, azo or anthraquinone dyes, and further natural dyes and pigments including carotenoids, xanthophyll, caramel, vegetal charcoal (Carbo vegetabilis), and melanins.

The invention also refers to the use of pigments in pharmaceutical or food manufacturing and in agriculture, e.g. seed coating/coloring.

Finally, the inventive pigments may be also be applied in industrial application, thereby including: coatings and inks (appliance and architectural coatings, automotive refinishing, custom finishing, industrial coating); leather coating; electronic product housings, plastics and rubber (toys and sporting goods, plastic packaging); textile coatings and inks.

### EXAMPLES

### Materials

The materials used in the preparation of the pigments of invention are listed in Table I

TABLE I

| Type | Compound | Brand/Type | Supplier; Origin |
|---|---|---|---|
| Cᵢ | Titanium dioxide TiO₂ | Tioxide RHD2 ™ | Tioxide Europe; Follonica (IT) |
| Cᵢ | Zinc oxide ZnO | — | Alfa Aesar J. M.; Karlsruhe (DE) |
| Cᵢᵢ | Iron oxide black Fe₃O₄ | Diploxide ™ 88P | Rockwood Specialties; Turin (IT) |
| Cᵢᵢ | Iron oxide red Fe₂O₃ | Diploxide ™ 226P | Rockwood Specialties; Turin (IT) |
| Cᵢᵢ | Iron oxide yellow FeOOH | Diploxide ™ 510P | Rockwood Specialties; Turin (IT) |
| Cᵢᵢ | Mica-TiO₂—SnO₂ | Timiron ™ Silk Gold | Merck; Darmstadt (DE) |
| Aᵢ | Quebracho tannin | QS-SOL | Silvachimica; S. Michele Mondovì (IT) |
| Aᵢ | Gallotannic acid | — | Shineway; Changsha, Hunan (China) |
| Aᵢ | Vegetal melanin | Phytomelanin ™ | Dr. Ghisalberti; Milan (IT) |
| Aᵢᵢ | Rutin 3H₂O | — | PVP; Parnaiba (Brazil) |
| Aᵢᵢ | Hesperidin 90% | — | Palmetto; Lakeland, FL (USA) |
| Aᵢᵢ | Anthocyanin 50% | Enocianina, Aldein ™ | Fornaciari; Reggio Emilia (IT) |
| Aᵢᵢᵢ | Curcumin 95% | Curcumin C³ Complex ™ | Sochim Intern.; Milan (IT) |
| Aᵢᵢᵢ | Gallic acid | — | Shineway; Changsha, Hunan (China) |
| Aᵢᵢᵢ | Ellagic acid | — | Shineway; Changsha, Hunan (China) |
| M | ZnCl₂•6H₂O | — | Sigma-Aldrich; Milan (IT) |
| M | CaCl₂•2H₂O | — | Sigma-Aldrich; Milan (IT) |
| M | AlCl₃•6H₂O | — | Sigma-Aldrich; Milan (IT) |
| M | Al₂(SO₄)₃•6H₂O | — | Sigma-Aldrich; Milan (IT) |
| M | MgCl₂•6H₂O | — | Sigma-Aldrich; Milan (IT) |
| M | NaAlO₂ | — | Univar; Turin (IT) |
| M | Na₂TiO₃ | — | Univar; Turin (IT) |
| M | TiCl₄ | — | Univar; Turin (IT) |
| Bᵢ | Lauric acid | Kortacid ™ 1299 | Akzo Nobel; Arese (IT) |
| Bᵢ | Palmitic acid | Kortacid ™ 1698 | Akzo Nobel; Arese (IT) |
| Bᵢ | Stearic acid | Kortacid ™ 1898 | Akzo Nobel; Arese (IT) |
| Bᵢ | Disodium stearoyl | Plantapon ™ ACG 35 | Cognis; Dusseldorf (DE) |
| | glutamate | | |
| Bᵢ | Nε-lauroyl-L-lysine | Amihope LL | Ajinomoto; Tokyo (JP) |
| Bᵢᵢ | Stearyl phosphoric acid | Knapsack ™ MDST | Clariant; Frankfurt (DE) |
| Bᵢᵢ | Octyl phosphoric acid | Knapsack ™ MDAH | Clariant; Frankfurt (DE) |
| Bᵢᵢᵢ | Sodium N-lauryl | Sarksyl ™ | Sigma-Aldrich; Milan (IT) |
| | sarcosinate | | |
| Bᵢᵥ | C₈-C₁₈ Perfluoroalkyl | AG530 ™ | Asahi Glass; Thornton Cleveleys (UK) |
| | phosphate ester DEA salt | | |

### Bᵥ—Preparation of Sodium Siloxane-Succinate

Succinic anydride (1.6 g, 16 mmol) and aminopropyl terminated polydimethylsiloxane DMS-A11 (7.2 g, 8 mmol) were dissolved in 200 ml dry methylene chloride and triethylamine (10 mL) was added. The solution was stirred under nitrogen at room temperature overnight. The organic phase was washed with 1M HCl (2×150 mL) and water (4×150 mL). After drying over anhydrous sodium sulfate the solvent was removed in vacuo, yielding a pale yellow oil (8.36 g, 95%). The corresponding sodium salt (“Sodium siloxane-succinate”) was obtained with the addition of 1 eq. NaOH 1N in water.

### Methods

The sequence of ion-exchange reactions uses preformed solutions of the reactants.

A⁽⁻⁾ salts—Preparation of Polyphenol Alkaline Solutions

The alkaline solutions of the polyphenols were standardized as 10% w/v solution in a 1N aqueous alkali, e.g. by dissolving 10 g of a polyphenol in 100 ml of in NaOH or KOH 1N at ambient temperature under stirring. Exemplary, disodium rutinate were freshly prepared by the addition of 5 g of rutin trihydrated in 100 ml of aqueous NaOH 1N to afford a transparent orange solution. Only Phytomelanin™ C was ready available as 10% w/v alkaline solution in NaOH 1N.

### B⁽⁻⁾ Salts—Preparation of the Surfactant Alkaline Solutions

Similarly, the alkaline solutions of the anionic surfactants were standardized as 5% w/v solution. When ready available as alkaline salt, they were separately prepared by dissolving 10 g of the alkaline salt of the surfactant in 200 ml of water. When the surfactant were available in the acidic form, the alkaline salt were prepared in situ by the reaction with 1 or 2 molar equivalent NaOH or KOH. Exemplary, sodium stearate was freshly prepared by the addition of 7.5 g of stearic acid and 2.64 ml of KOH 1N in 150 ml of water, then melted at around 80° C. to afford a gelled solution of sodium stearate. The suspension of the high-melting alkaline salts were warmed up to a temperature at which the solution is in a fluid, pourable form.

### M⁽⁺⁾ Salts—Preparation of the Solution of Acidic, Cationic Salts

The solution of soluble salt of polyvalent cations (e.g. M²⁺X₂ solution”; M²⁺=earthy-alkaline cation) or (“M³⁺X₃ solution”; e.g. Me³⁺=Al³⁺) or (“M⁴⁺X₄ solution”; e.g. Me⁴⁺=Ti⁴⁺) were prepared and stoked as 1M solution by dissolving 1 molar equivalent in 1 litre of demineralised water. Optionally, these solutions were prepared as 1:1 mol/mol mixture with an inorganic acid (e.g. HCl)

### M⁽⁻⁾ Ampho Salts—Preparation of the Alkaline Solution of Amphoteric Salts

Similarly, the solution of soluble amphoteric salts of the trivalent or tetravalent cationic metal, namely sodium aluminate and sodium titanate were prepared and stocked as 1M solution, e.g. dissolving 1 molar equivalent of NaAlO₂ (82 g) or Na₂TiO₃ (141.8 g), respectively, in 1 litre of demineralised water.

Method I—Preparation of C-Mₐ-A-M_{b}-B Pigments with the Use of M⁽⁺⁾ Salts

Into a 1 litre beaker fitted with a mechanical stirrer were suspended 200 g of a pigment (C) in 500 ml of water, then 80 meq of a M⁽⁻⁾ salt solution (e.g. 40 ml of CaCl₂ 1M, or 30 ml of AlCl₃ 1M, or 20 ml of TiCl₄ 1M) were added. A pH from 3 to 5 is attained. The slurry was warmed at 80-85° C. and stirred for 25 minutes, then added dropwise with 80 ml of 10% w/v A⁽⁻⁾ salt solution, thereby stirred for further 15 minutes at the same temperature. Thereafter, the slurry was slowly added with 150 ml a warm solution (80° C.) containing 5% w/v of Basalt and stirred for 20 minutes. The resulting suspension having a pH from 7.5 to 9 was neutralized (pH ˜7) with 26 meq. of a M⁽⁻⁾ salt solution.

Method II—Preparation of C-Mₐ-A-M_{b}-B Pigments with the Use of M⁽⁻⁾ Ampho Salts

Into a 1 litre beaker fitted with a mechanical stirrer are suspended 200 g of a pigment (C) in 500 ml of water. The slurry was warmed at 80-85° C., then added dropwise with 50 ml solution containing 1M M⁽⁻⁾ ampho salt solution (e.g. NaAlO₂, Na₂TiO₃, NaZn(OH)₃). Successively there were added with 40 ml of 10% w/v A″salt solution and NaOH until pH of 11 was attained. The slurry was kept under stirring for 30 minutes, after which there was started a slow acidification with HCl 3.5N. Once there was attained a 10.5 pH, 60 meq of a M⁽⁺⁾ salt solution (e.g. 30 ml of CaCl₂ 1M, or 20 ml of AlCl₃ 1M) were added were added in 10 minutes at 80° C. After 20 minutes, the slowly acidification with HCl 3.5N was carried out until attaining a pH of 7. Then 150 ml of a warm solution (80° C.) containing 5% w/v of B″salt was slowly added while maintaining pH ˜7 by adding a M⁽⁺⁾ salt solution.

Method III—Preparation of C-A-M_{b}-B Pigments with the Use of M⁽⁺⁾Salts

Into a 1 litre beaker fitted with a mechanical stirrer are suspended 200 g of a pigment (C) in 500 ml of water, then the slurry was warmed at 80-85° C. There was started the addition of 80 ml of 10% w/v A⁽⁻⁾ salt solution, thereby stirred for further 15 minutes at the same temperature. Thereafter, the slurry was slowly added with then 80 meq of a Me⁽⁻⁾ salt solution (e.g. 40 ml of CaCl₂ 1M, or 30 ml of AlCl₃ 1M, or 20 ml of TiCl₄ 1M) and stirred for 10 minutes. Then the slurry is added with 150 ml of a warm solution (80° C.) containing 5% w/v of Basalt and stirred for 20 minutes. In case of monovalent B⁽⁻⁾ salts, the resulting pH was approximately neutral, in case of dianionic B⁽²⁻⁾ salts the resulting suspension was neutralized (pH ˜7) with an equivalent of a M⁽⁺⁾ salt solution.

### Examples A-AS

A variety of pigments comprising an inner core made of inorganic oxides and hydrated oxides, an inner layer of plant polyphenol and an outer layer of hydrophobic substances were prepared by Method I, II or III as illustrated in Table III.

TABLE III
Pigment according to the invention

| Example | | | | | | |
|---|---|---|---|---|---|---|
| No. | C | Mₐ | A | M_{b} | B | Method |
| A | Fe₂O₃ | | Gallic | Al | C₈₋₁₈-perfluoro phosphate | III |
| B | Fe₂O₃ | Al | Rutin | Zn | Palmitate | I |
| C | Fe₂O₃ | Al | Curcumin | Al | Stearate | I |
| D | Fe₂O₃ | Ca | Proanthocyanidin | Zn | Stearate | I |
| E | Fe₂O₃ | Ca | Ellagic | Al | Stearoyl glutamate | II |
| F | Fe₂O₃ | Al | Rutin | Al | Stearoyl glutamate | II |
| G | Fe₃O₄ | Al | Gallic | Al | Laurate | I |
| H | Fe₃O₄ | Ca | Quebracho tannin | Ca | Laurate | I |
| I | Fe₃O₄ | | Gallic | Al | Nε-Lauroyl-L-lysinate | III |
| J | Fe₃O₄ | Ca | Ellagic | Al | Stearoyl glutamate | II |
| K | Fe₃O₄ | Al | Rutin | Al | Stearoyl glutamate | II |
| L | FeOOH | | Gallic | Al | N-Lauryl sarcosinate | III |
| M | FeOOH | Zn | Phytomelanin | Mg | Stearate | I |
| N | FeOOH | Mg | Rutin | Mg | Stearate | I |
| O | FeOOH | Ca | Ellagic | Al | Stearoyl glutamate | II |
| P | FeOOH | Al | Rutin | Al | Stearoyl glutamate | II |
| Q | Mica-TiO₂—SnO₂ | Ca | Anthocyanin | Al | Stearoyl glutamate | II |
| R | TiO₂ | Ti | Phytomelanin | Al | C₈₋₁₈-perfluoro phosphate | II |
| S | TiO₂ | | Phytomelanin | Al | C₈₋₁₈-perfluoro phosphate | III |
| T | TiO₂ | Zn | Phytomelanin | Zn | Nε-Lauroyl-L-lysinate | II |
| U | TiO₂ | | Phytomelanin | Zn | Nε-Lauroyl-L-lysinate | III |
| V | TiO₂ | Ca | Anthocyanin | Al | Stearoyl glutamate | II |
| W | TiO₂ | Al | Phytomelanin | Al | Stearoyl glutamate | II |
| Y | TiO₂ | | Phytomelanin | Al | N-Lauryl sarcosinate | III |
| Z | TiO₂ | Ca | Anthocyanin | Al | Octyl phosphate | II |
| AA | TiO₂ | Zn | Phytomelanin | Ca | Octyl phosphate | II |
| AB | TiO₂ | | Phytomelanin | Ca | Octyl phosphate | III |
| AC | TiO₂ | Ca | Anthocyanin | Al | Siloxane succinate | II |
| AD | TiO₂ | Al | Phytomelanin | Ca | Siloxane succinate | II |
| AE | TiO₂ | | Phytomelanin | Ca | Siloxane succinate | III |
| AF | TiO₂ | Ca | Ellagic | Zn | Stearate | I |
| AH | TiO₂ | Ca | Hesperidin | Al | Stearate | I |
| AI | TiO₂ | | Phytomelanin | Zn | Stearate | III |
| AJ | TiO₂ | Ti | Phytomelanin | Al | Stearate | II |
| AK | TiO₂ | Al | Phytomelanin | Al | Stearoyl glutamate | II |
| AL | TiO₂ | | Phytomelanin | Al | Stearoyl glutamate | III |
| AM | TiO₂ | Zn | Phytomelanin | Ca | Stearyl phosphate | II |
| AN | TiO₂ | | Phytomelanin | Ca | Stearyl phosphate | III |
| AO | ZnO | Zn | Ellagic | Zn | C₈₋₁₈-perfluoro phosphate | I |
| AP | ZnO | Ca | Gallotannin | Ca | Laurate | I |
| AQ | ZnO | Zn | Ellagic | Zn | Nε-Lauroyl-L-lysinate | I |
| AR | ZnO | Zn | Ellagic | Zn | N-Lauryl sarcosinate | I |
| AS | ZnO | Zn | Ellagic | Zn | Stearoyl glutamate | I |

The resulting pigments comprises from about 2 to 4% (w/w) of inner layer A and from about 3 to 5% (w/w) of the outer layer B, the remaining being the inorganic core C.

### Recovery of the Pigments

The slurry is filtered and the filtrate is washed with warm deionized water until the soluble salts are removed. In the case of dry pigments, the resulting cake was dried at 80° C. and grinded. In the case of wet pigments, the wet cake is added with pigments dispersants such as Arlacel P100 (ICI-Bregaglio, Milan, IT) until a suspension of 30% solids was attained.

### Test Example 1-2

### In Vitro Evaluation on Attenuation of the Photodynamic Properties

The UVA exposure of the pigment of invention versus uncoated pigments was evaluated by the in vitro Phototoxicity 3T3 NRU Phototoxicity test, according to the official method issued on the EC Official Journal Aug. 6, 2000.

Briefly, the pigments are dissolved in PBS at concentrations between 0.039 and 2.5 mg/ml. Permanent mouse fibroblasts, Balb/c 3T3, cl31, from American type culture collection (ATCC) were seeded at 15.000 cell/well in DMEM (Dulbecco modified essen. medium) supplemented with 10% FCS (fetal calf serum) with 4 mM glutamine, penicillin, and streptomycin. The Balb/c 3T3 cells were kept in culture for 24 h (7.5% CO₂, 37° C.) until half-confluent monolayer. Non treated cells (negative controls) were tested concurrently with each in vitro phototoxicity assay. Thereafter one of the two plates is exposed for 11′ to a non-cytotoxic UVA light dose of 5 J/cm (1.7 mW/cm²).

The lamp used in the experimenters is a solar light simulator, at constant emission in the UVA range (315-400 nm). After two washing with PBS, culture medium is added and cells are incubated (7.5% CO₂, 37° C.) overnight (18-22 h). NR medium was prepared adding Neutral Red solution 0.33% in PBS to the DMEM to attain a final conc. of 50 μg/ml. Cells were washed with PBS and 100 μl of NR medium are then added to each well. The cells are incubated for 3 h at 37° C., 7.5% CO₂. Afterwards, the medium was discharged, the cells were washed and the plates dried. 150 μl of acetic acid solution 1% in ethanol:water 1:1 v/v were added in each well, shaking for 15′ to ensure NR extraction and the formation of a homogeneous solution. The optical density of NR extract was measured at 540 nm with a spectrophotometer.

The results are illustrated in Table II

TABLE II
Phototoxic action on UV-treated fibroblast in the presence
of the pigment of invention compared to untreated pigments.

| | % Decrease of | % Decrease of |
|---|---|---|
| | the phototoxic | the phototoxic |
| | behaviour at | behaviour at |
| | conc. 2.5 mg/ml | conc. 1.25 mg/ml |
| Pigment of the Example D | 15% | 32% |
| Pigment of the Example B | 3% | 9% |
| Pigment of the Example M | 6% | 14% |
| Pigment of the Example AF | −3% | 22% |
| Pigment of the Example AH | 11% | 30% |
| Pigment of the Example G | 4% | 27% |
| Pigment of the Example N | 9% | 31% |
| Pigment of the Example C | 13% | 40% |
| Pigment of the Example AP | 4% | 32% |

### Cosmetic Applicative Examples

### Applicative Examples 1-3

### Foundations

| | Appl. | Appl. | Appl. |
|---|---|---|---|
| 100 g of foundation contain: | Ex. 1 | Ex. 2 | Ex. 3 |
| Triethanolamine Stearate | 2.5 | 2.5 | 2.5 |
| Mono- and di-Glycerol Stearate | 0.4 | 0.4 | 0.4 |
| Talc (magnesium silicate) | 1.9 | 1.9 | 1.9 |
| Pigment of Example A | 3.7 | 3.1 | 2.9 |
| Pigment of Example O | — | 0.7 | 0.8 |
| Pigment of Example J | 0.5 | 0.5 | 0.6 |
| Pigment of Example Z | 9.8 | 9.8 | 9.8 |
| Micronized Nylon | 9.0 | 9.0 | 9.0 |
| Mix of PEG-6 and PEG-32 | 9.0 | 9.0 | 9.0 |
| Cyclomethycone | 13.0 | 13.0 | 13.0 |
| Propylenglycol | 5.0 | 5.0 | 5.0 |
| Glycerol | 4.5 | 4.5 | 4.5 |
| Demineralized water | 25.6 | 25.6 | 31.6 |

### Applicative Examples 4-6

### Eye Shadows

| | Appl. | Appl. | Appl. |
|---|---|---|---|
| 100 g of emulsions for eyelids contain: | Ex. 4. | Ex. 5 | Ex. 6 |
| Carnauba Wax | 1.1 g | 1.1 g | 1.1 g |
| Partially Hydrogenated Soybean Oil | 1.9 g | 1.9 g | 1.9 g |
| Palm Oil | 1.9 | 1.9 | 1.9 |
| Triethanolamine Stearate | 5.0 | 5.0 | 5.0 |
| PEG-1000 | 13.0 | 13.0 | 13.0 |
| Talc (Magnesium silicate) | 0.7 | 0.7 | 0.7 |
| Pigment of Example E | 2.6 | 1.9 | 2.3 |
| Pigment of Example K | 2.0 | 2.5 | 1.8 |
| Pigment of Example P | 2.7 | 1.5 | 2.5 |
| Pigment of Example U | 3.0 | 3.0 | 3.0 |
| Sodium Polymethacrylate | 0.5 | 0.5 | 0.5 |
| Preservatives | 0.5 | 0.5 | 0.5 |
| Demineralized water | qb to 100 | to 100 | to 100 |

### Applicative Example 6

### Anti-Sun Cream

100 g of emulsions used as sun filters contain:

| | |
|---|---|
| Glycerin 87% | 3.0 g |
| Disodium EDTA | 0.2 g |
| Imidazolidinyl Urea | 0.3 g |
| Decyl Glucoside | 1.0 g |
| Xanthan Gum | 0.3 g |
| D-Panthenol in Propylene Glycol | 2.0 |
| Aqua demonizzata | q.b. (57) |
| Pigment of Example AK | 5.0 g |
| Trimethoxycaprylylsilane Ethylhexyl Methoxycinnamate | 7.5 g |
| Diethylamino Hydroxybenzoyl Hexyl Benzoate | 2.0 g |
| Cyclomethicone | 2.0 g |
| Ceteareth-6, Stearyl Alcohol | 3.5 g |
| Ceteareth-25 | 1.5 g |
| Bees Wax | 0.5 g |
| Cetearyl Alcohol | 3.0 g |
| Caprylic/Capric Triglyceride | 10.0 g |
| Tocopheryl Acetate | 1.0 g |
| Preservatives and perfume | q.s. |

1. A pigment having the following structure (I): 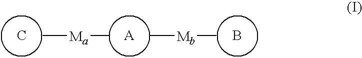
wherein:
C denotes the internal pigment core of formula [M^{x}(O)_{y}(OH)_{z}];
M^{x}=Ti^{IV}, Zn^{II}, Z^{IV}, Sn^{IV}, Ce^{IV}, Fe^{II}, Fe^{III}, Sn^{II}, Cr^{VI} or Mn^{II}
x=2, 3 or 4; and (y/2+z)=x;
said C having a diameter comprised between 20 and 0.02 mm;
A denotes a plant polyphenols having an active-redox, radical scavanger behaviour and at least two chelating moieties selected from:
Aᵢ: polymeric polyphenols;
Aᵢᵢ: anthocyanins or flavonoids;
Aᵢᵢᵢ: non-flavonoid plant polyphenols;
B denotes an outer hydrophobic layer constituted of one or more surfactant selected from:
Bᵢ: carboxylic-ended surfactants;
Bᵢᵢ: phosphoric-ended surfactants;
Bᵢᵢᵢ: sulfonic-ended surfactants;
Bᵢᵥ: perfluorurated surfactants;
Bᵥ: siloxane chelating surfactants.
Mₐ and M_{b} denotes, each independently, M^{q+};
where M^{q+}=Ca²⁺, Zn²⁺, Ti⁴⁺, Si⁴⁺, Al³⁺, Mg²⁺, or Sr²⁺;
a=0 or 1; b=1; q=2, 3 or 4;

2. The pigment according to claim 1 wherein a=0.

3. The pigment according to claim 1 wherein Aᵢ is selected from gallotannin, ellagitannin, complex tannin, condensed tannins, or a mixture thereof.

4. The pigment according to claim 1 wherein Aᵢ is a vegetal melanin obtained by oxidative copolymerization of vegetal monomers and eumelanin precursors.

5. The pigment according to claim 1 wherein Aᵢᵢ is an anthocyanin selected in the group comprising: pelargonidin, cyanidin, delphinidin, petunidin, peonidin, malvidin, and di and tri-saccharides, or 3-acylated derivatives thereof.

6. The pigment according to claim 1 wherein Aᵢᵢ is a flavonoid selected in the group comprising: dihydroquercetin, miricetin, luteolin, quempferol, morin, quercetin, esperetin, naringenin, fisetin, fustine, and a glycosides or esters thereof.

7. The pigment according to claim 1 wherein Aᵢᵢᵢ is a non-flavonoid plant polyphenol.

8. The pigment according to claim 7 wherein the non-flavonoid polyphenol is gallic acid or ellagic acid.

9. The pigment according to claim 1 wherein Bᵢ is selected in the group consisting of: carboxylate of formula ⁽⁻⁾ OOC—R; dicarboxylate of formula ⁽⁻⁾OOC—R—OOC⁽⁻⁾; a-hydroxycarboxylate of formula ⁽⁻⁾OOC—CHR′—O—CO—R; alkylsuccinamate of formula ⁽⁻⁾OOC—CH₂CH₂—O—CO—R; N-alkanoylsarcosinate or glycinate of formula ⁽⁻⁾OOC—CH₂—NR′—CO—R; N-acylglutamate of formula ⁽⁻⁾OOC—CH₂CH₂CH(NH—CO—R)—COO⁽⁻⁾; N-acylaspartate of formula ⁽⁻⁾OOC—CH₂CH(NH—CO—R)—COO⁽⁻⁾; and Ne-acyl-lysinate of formula ⁽⁻⁾OOC—CH(NH₂)—(CH₂)₄—NH—CO—R.

10. The pigment according to claim 1 wherein Bᵢᵢ is selected in the group consisting of: alkyl o dialkyl-phosphate of formula: ⁽⁻⁾OₐP(O)—(OR)_{b}; wherein a=1 or 2; and b=3-a.

11. The pigment according to claim 1 wherein Bᵢᵢᵢ is selected in the group consisting of: alkyl sulfonate of formula ⁽²⁻⁾O₃S—R; alkylsulfate of formula ⁽²⁻⁾O₃S—O—R; alkyl ester sulfonate of formula: ⁽²⁻⁾O₃S—CHR—COOR′; alkyl amide sulfate of formula ⁽²⁻⁾O₃SO—NHR′—CO—R; alkyl-isethionate of formula: ⁽²⁻⁾O₃S—CH₂CH₂—CO—R; N-acyl-N-alkyltaurate of formula ⁽²⁻⁾O₃S—CH₂CH₂—NR′—CO—R; C₉-C₂₀ alkylbenzene-sulfonate; alkylsulfosuccinate of formula ⁽²⁻⁾O₃S—CH(COOR′)—CH₂—R sulfosuccinate monoesters or diesters; alkylglycosides sulphate, and the like.

12. The pigment according to claim 9, 10 or 11 wherein each —R or —C—OR radical is a saturated or unsaturated, linear or branched, alkyl or acyl C₂-C₂₂, preferably C₈-C₁₈.

13. The pigment according to claim 12 wherein the —R or —C—OR radicals is derived from capric, caprylic, capric, lauric, myristic, palmitic, stearic, isostearic, sebacic, palmitoleic, linoleic acids and alcohols, alone or in combination.

14. The pigment according to claim 13, wherein said R comprises a polyalkoxy chain of formula —(OCH₂CHR′)_{z}—O— with z comprised between 1 and 100, between 1 and 10; and each R′ is H or C₁-C₃alkyl, preferably C₁.

15. The pigment according to claim 1 wherein Bᵢᵥ is selected in the group consisting of: perfluoroalkylcarboxylate of formula ⁽⁻⁾OOC—R″, perfluoro-alkyl or dialkyl-phosphate of formula: (−) OₐP(O)—(OR″)_{b}; a=1 or 2; b=3; perfluoroalkylsulfonate of formula ⁽⁻⁾O₃S—R″; wherein R″ is a fluorocarburic C₄-C₂₀ chain, preferably C₈-C₁₈;

16. The pigment according to claim 1 wherein Bᵥ is a silicone polymer comprising a hydrophobic polysiloxane backbone and at least one metal binding site which is covalently bound to the hydrophobic polysiloxane backbone.

17. A method of manufacturing a pigment according to claim 1 wherein a suspension of core pigment (C) in water is sequentially reacted with an alkaline solution of A, and then with an alkaline solution of B.

18. The method according to claim 17 wherein the core pigment (C) is reacted with i) a soluble salt of M^{q+}; ii) an alkaline solution of A; iii) a soluble salt of M^{q+}, and iv) an alkaline solution of B; to afford a pigment with schematic structure (I-a):

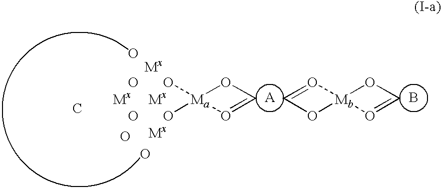

19. The method according to claim 17 wherein the core pigment (C) is reacted with i) an alkaline solution of A; ii) a soluble salt of M^{q+}, and iii) an alkaline solution of B; to afford a pigment with a schematic structure (I-b):

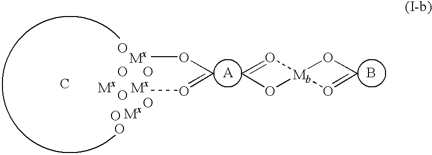

20. A cosmetic composition intended for make-up comprising a pigment according to claim 1 in an amount between 3% to 80% by weight.

21. The cosmetic composition intended for solar protection comprising a pigment according to claim 1 in an amount between 1% and 20% by weight.

22. The cosmetic composition according to claim 20 or 21 in the form of foundation, pressed powder, face powder, lipstick, eye shadow, eyebrow pencil, eye liner, mascara, anhydrous or hydrated emulsion, and paste.

23. A pharmaceutical or food product comprising a pigment according claim 1 in an amount between 3% to 80% by weight.
